(19) United States
(12) Patent Application Publication
(10) Pub. No.: US 2005/0159614 A1
(43) Pub. Date: Jul. 21, 2005

(54) Norbornane based cycloaliphatic compounds containing nitrile groups

(76) Inventors: Alan Martin Allgeier, Wilmington, DE (US); Christian Peter Lenges, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/760,779

(22) Filed: Jan. 19, 2004

(51) Int. Cl. C07C 255/47

(52) U.S. Cl. 558/429

(57) ABSTRACT

This invention relates to novel norborane nitrile derivatives, and corresponding methods for making the same using hydrocyanation reactions.

### FIELD OF THE INVENTION

The present invention discloses novel norbornane based nitrile derivatives as well as a method for making them comprising hydrocyanation reactions.

### BACKGROUND OF THE INVENTION

Cycloaliphatic compounds containing nitrile groups are of great interest as precursors to a variety of useful molecules with applications as intermediates for the production of polymers, as fragrance intermediates or as intermediates for life science applications. These nitrile functional groups can be converted to novel amines, carboxylic acids, or alcohol groups. Methylene amine compounds derived from nitrile compounds, for instance, can be used as epoxy curing agents, either neat or as the adducted form. One skilled in the art of epoxy formulation will select different curing agents based on their structure to control curing time, pot life and physical properties of resulting coatings, adhesives, castings or composites. There is great interest in the economic preparation of cycloaliphatic amine compounds from nitrile compounds bearing different functional groups for epoxy cure applications.

U.S. Pat. No. 2,956,987 describes the preparation of the norbornane derivative nitrilo-norcamphane carboxylic acid. JP 06184082 describes the preparation of norcamphane-dicarbonitrile. A palladium catalyzed route to norcamphane-dicarbonitrile is described in the Preprints of the American Chemical Society, Division of Petroleum Chemistry (1969), 14(2), B29-B34. The preparation of the norbornane derivative dicyanotricyclodecane is described in U.S. Pat. No. 4,151,194. GB1480999 describes the preparation and use of nirtile derived triamines based on the norbornane skeleton as isocyanate precursors for polyurethane lacquer formation but fails to suggest the novel structures suggested herein.

Prior to the present invention the norbornane dicarbonitrile was known as a precursor to useful monomers but there has been little work on extending the basic norbornane skeleton to substituted derivatives, of the kind described herein, to control properties and reactivity of such derivatives. The inventors have discovered that unique advantages can be achieved regarding the physical properties and the reactivity of norbornane nitrile derivatives if these norbornane derivatives are prepared with additional substituents at the norbornane core.

Prior to the present invention, it was not known that the norbornene derivatives of this invention could be converted selectively in a hydrocyanation process to norbornane derivatives with nitrile groups. There is a need to access these novel cycloaliphatic hydrocarbons, which have one or more functional groups, such as nitriles, amines, alcohols or carboxylic acids and which are substituted by additional alkyl or aryl substituents. Especially cycloaliphatic hydrocarbons with two and more than two functional groups are of interest.

Thus, there is a need for norbornane derivatives, which contain nitrile groups. There also remains a need for a method to produce such norbornane derivatives, which contain nitrile groups. These needs are met by the present invention.

### SUMMARY OF THE INVENTION

Accordingly, it is one object of the present invention to provide novel norbornane derivatives containing nitrile groups. It is another object of the present invention to provide a method for preparing such norbornane compounds. These and other objects will become apparent in the following detailed description.

### DETAILED DESCRIPTION OF THE INVENTION

The present invention therefore provides novel norbornane derivatives containing nitrile groups of formula (I): 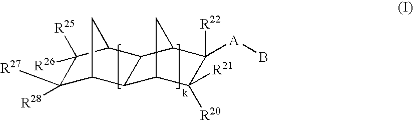
either alone, as combinations of these, and/or as mixture of isomers of these,
wherein
k=0, 1 or 2 and the bridging CH₂ group may be on the same or opposite side with respect to the first bridging CH₂ group,
   wherein
R²⁰, R²¹, R²² can be the same or different and are each independently H, a C₁ to C₂₀ alkyl group, a C₁ to C₂₀ alkyl group substituted with a hydroxyl, a C₁ to C₁₈ perfluoroalkyl group, a phenyl group, an C₆ to C₂₀ aryl group substituted with a C₁-C₁₂ alkyl group, an C₆ to C₂₀ aryl group substituted with a hydroxyl group, a C(O)OR²⁹ group (with R²⁹ selected to be a C₁ to C₂₀ linear or branched or cyclic alkyl or C₆ to C₂₀ aryl group), or an alkylene chain (—(CH₂)_{q}—; q equals an integer 0-16) or nothing (in which case A or B may connect back to the norbornane skeleton) with the proviso that R²⁰, R²¹ and R²² do not comprise a cyano group or an amino group and
   wherein
A equals nothing or any alkylene chain (—(CH₂)ₚ—; p equals an integer 1-16), any substituted C₁ to C₂₀ alkylene group (provided the substituent does not comprise a cyano group or an amino group and does not interfere with the process of this invention), a C₁ to C₂₀ hydrocarbyl or cyclohydrocarbyl group that may comprise one or more alkene groups or a C₁ to C₁₈ perfluoroalkylene group, and wherein A may form a ring of greater than 5 carbons that connects to the norbornane skeleton through R²⁰, R²¹ or R²²
with the proviso that R²⁰, R²¹ or R²² cannot all be H if A equals nothing and
   wherein
B equals —CN, —(CH₂)ₛOH or —C(O)OR²⁴
   with s equal to an integer 0-12 and with R²⁴ selected to be H, a C₁ to C₂₀ linear or branched or cyclic alkyl or alkylene group, a C₆ to C₂₀ aryl group or a C₁ to C₁₈ perfluorinated alkyl group and wherein R²⁴ may connect to the norbornane skeleton through R²⁰, R²¹ or R²²
   or wherein
   R²⁴ may equal a —C(O)— group which connects to the norbornane skeleton through R²⁰, R²¹ or R²² forming a cyclic anhydride. and
      wherein
R²⁵, R²⁶, R²⁷, R²⁸ can be the same or different and are each independently H or —CN, with the proviso that only one of R²⁵, R²⁶, R²⁷, R²⁸ is —CN.

The relative spatial orientation of the substituents on the norbornane skeleton can be any possible combination. Stereoisomers are common embodiments of the invention.

These compounds are of interest as precursors to a variety of useful molecules with applications as intermediates for epoxy cure applications, the production of polymers, as fragrance intermediates or as intermediates for life science applications.

The inventors have discovered that certain norbornene derivatives can be contacted with hydrogen cyanide, in the presence of a catalyst and optionally a promoter at a temperature of about −25° C. to about 200° C. to yield norbornane nitrile derivatives of the formula (I), wherein the catalyst comprises a transition metal, preferably palladium or nickel and an organic phosphorous ligand.

Thus the present invention also provides a hydrocyanation method for preparing norbornane derivatives, which contain nitrile groups. Generally, the present method yields the present norbornane nitrile derivatives as a mixture of isomers. This mixture of isomers generally does not contain the isomers of this invention in approximately equal amounts. Instead, the method yields several isomeric compounds as main products. The isomer favored in this method is a function of process conditions and/or the type of catalyst or catalysts used and/or the type of ligand used and/or the use of an optional promoter. However, it is to be understood that both the individual compounds and also the mixtures of isomers thereof are within the scope of the present invention.

The method for making the compounds of the present invention involves a hydrocyanation process with the use of a ligand and a Group VIII metal or compound. Optionally, one may use a Lewis acid in the hydrocyanation process as a promoter, and may optionally use a solvent.

Generally, a Group VIII metal or compound thereof is combined with at least one ligand to provide the catalyst. Among the Group VIII metals or compounds, nickel, cobalt, and palladium compounds are preferred to make the hydrocyanation catalysts. A nickel or palladium compound is more preferred. For example, a zero-valent nickel compound that contains a ligand that can be readily displaced by another, more desired ligand as described in the prior art is the most preferred source of Group VIII metal or Group VIII metal compound.

Zero-valent nickel compounds can be prepared or generated according to methods known in the art. Three preferred zero-valent nickel compounds are Ni(COD)₂ (COD is 1,5-cyclooctadiene), Ni(P(O-o-C₆H₄CH₃)₃)₃ and Ni{P(O-o-C₆H₄CH₃)₃}₂(C₂H₄); these are known in the art.

Alternatively, divalent nickel compounds can be combined with a reducing agent, to serve as a source of zero-valent nickel in the reaction. Suitable divalent nickel compounds include compounds of the formula NiX²₂ wherein X² is halide, carboxylate, or acetylacetonate. Suitable reducing agents include metal borohydrides, metal aluminum hydrides, metal alkyls, Li, Na, K, Zn, Al or H₂. Elemental nickel, preferably nickel powder is also a suitable source of zero-valent nickel.

Suitable ligands for the present invention are monodentate and/or bidentate phosphorous-containing ligands selected from the group consisting of phosphites or phoshinites or phosphines. Preferred ligands are monodentate and/or bidentate phosphite ligands.

The preferred monodentate and/or bidentate phosphite ligands are of the following structural formulae: 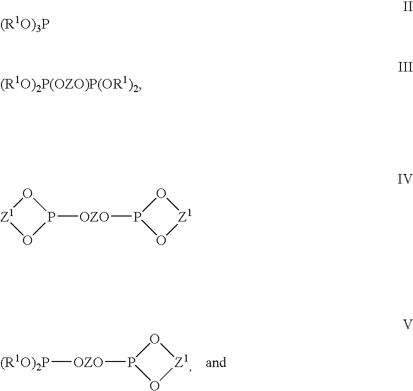
In formulae II, III, IV and V, R¹ is phenyl, unsubstituted or substituted with one or more C₁ to C₁₂ alkyl or C₁ to C₁₂ alkoxy groups; or naphthyl, unsubstituted or substituted with one or more C₁ to C₁₂ alkyl or C₁ to C₁₂ alkoxy groups; and Z and Z¹ are independently selected from the group consisting of structural formulae VI, VII, VIII, IX, and X: 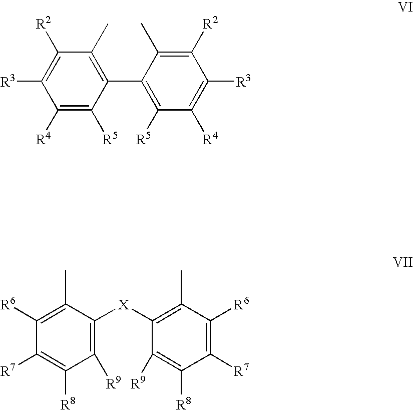
wherein
R², R³, R⁴, R⁵, R⁶, R⁷, R⁸, and R⁹ are independently selected from H, C₁ to C₁₂ alkyl, and C₁ to C₁₂ alkoxy;
X is O, S, or CH(R¹⁰); R¹⁰ is H or C₁ to C₁₂ alkyl; 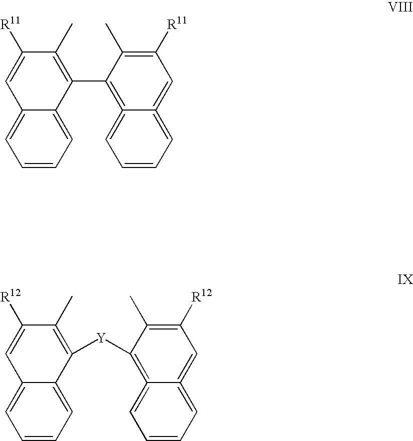
   wherein
R¹¹ and R¹² are independently selected from H, C₁ to C₁₂ alkyl, and C₁ to C₁₂ alkoxy; and CO₂R¹³,
R¹³ is C₁ to C₁₂ alkyl or C₆ to C₁₀ aryl, unsubstituted or substituted. with C₁ to C₄ alkyl
Y is O, S, CH(R¹⁴);
R¹⁴ is H or C₁ to C₁₂ alkyl 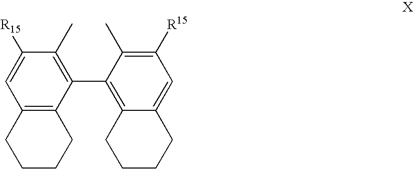
   wherein
R¹⁵ is selected from H, C₁ to C₁₂ alkyl, and C₁ to C₁₂ alkoxy; and CO₂R¹⁶,
R¹⁶ is C₁ to C₁₂ alkyl or C₆ to C₁₀ aryl, unsubstituted or substituted with C₁ to C₄ alkyl.

In the structural formulae II through X, the C₁ to C₁₂ alkyl, and C₁ to C₁₂ alkoxy groups may be straight chains or branched.

Examples of bidentate phosphite ligands that are useful in the present process include those having the formulae XI to XXXIV, shown below wherein for each formula, R¹⁷ is selected from the group consisting of H, methyl, ethyl or isopropyl, and R¹⁸ and R¹⁹ are independently selected from H or methyl:

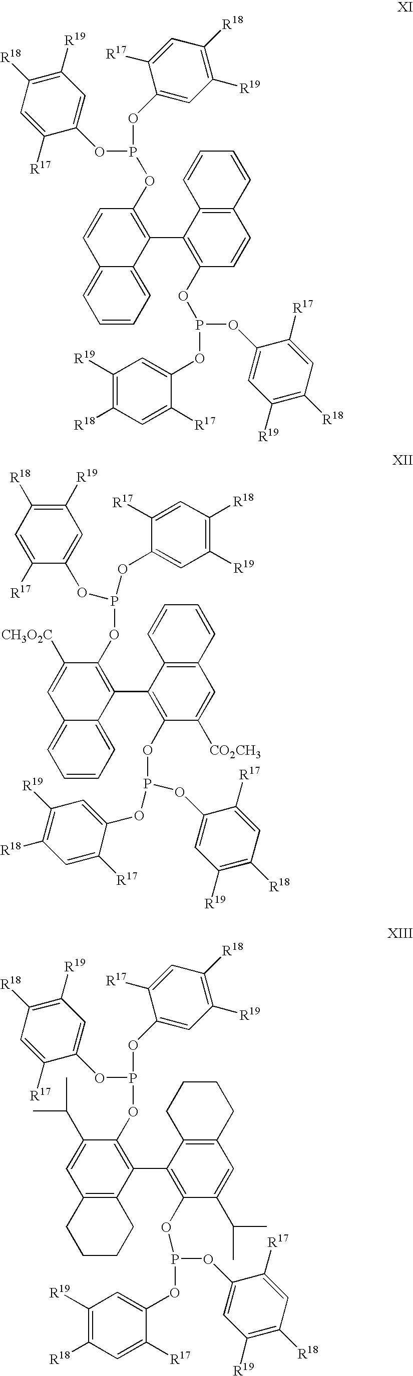

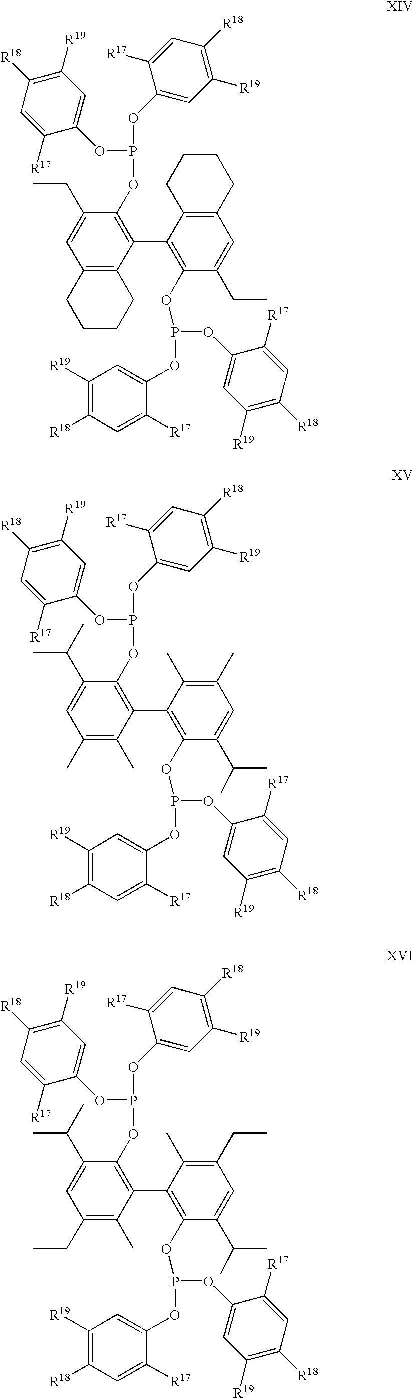

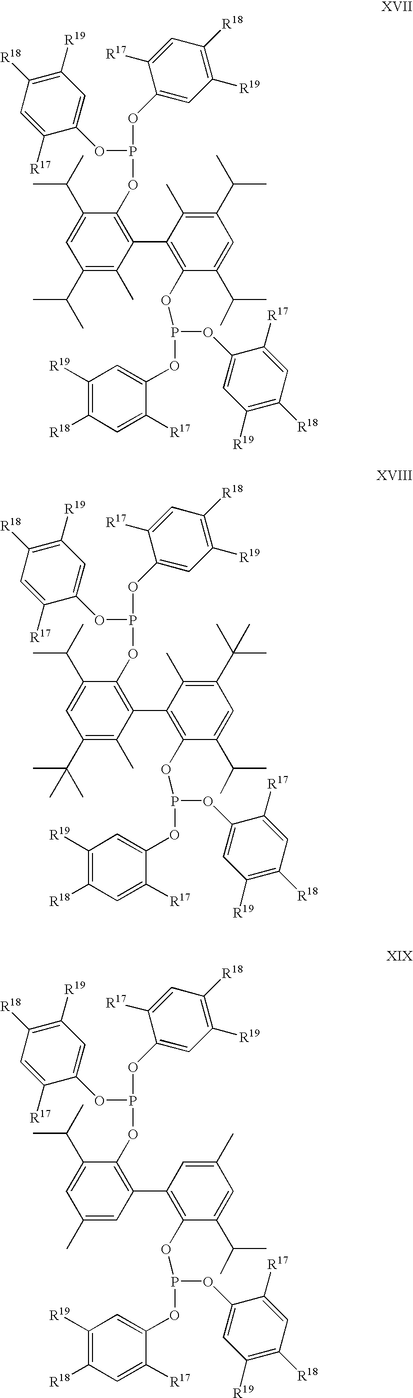

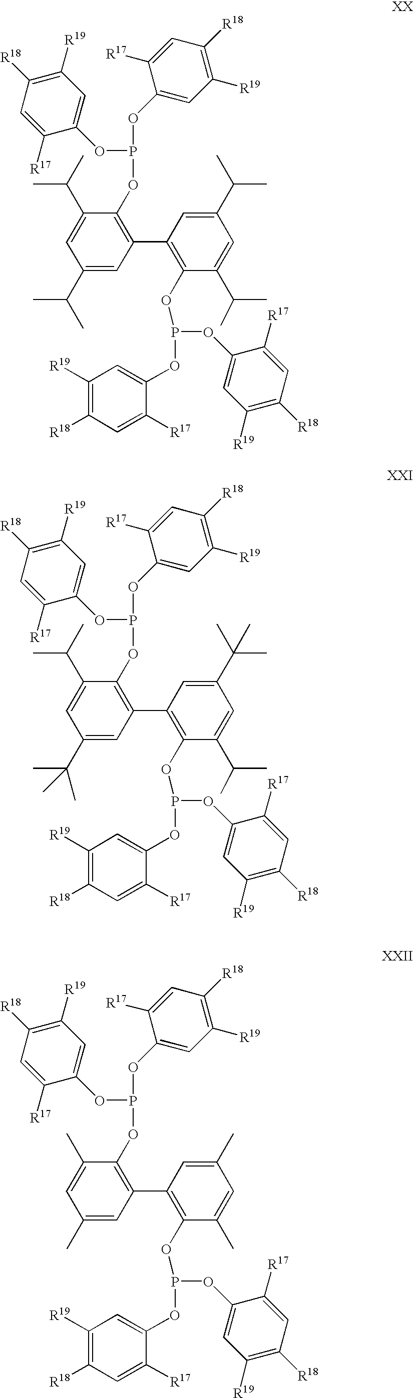

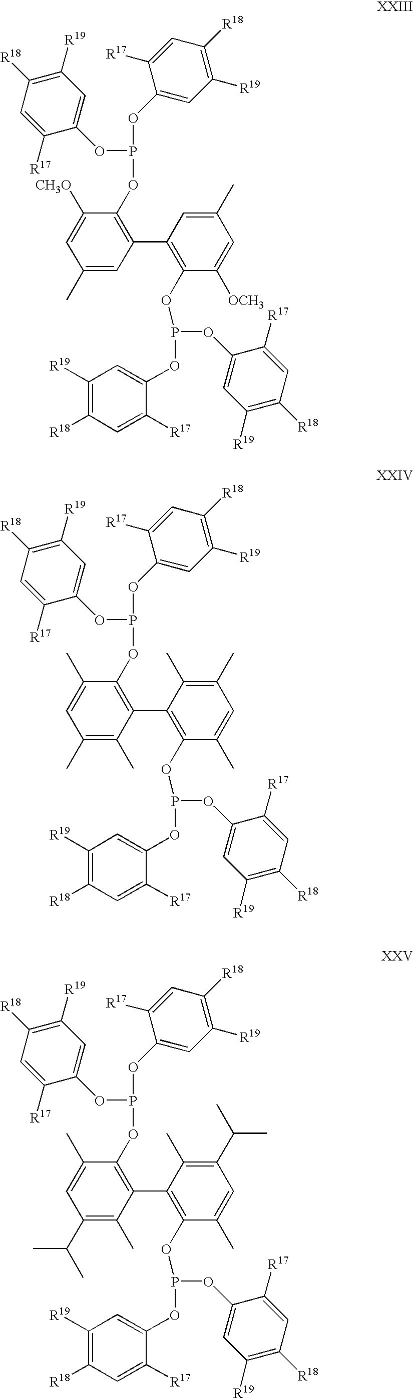

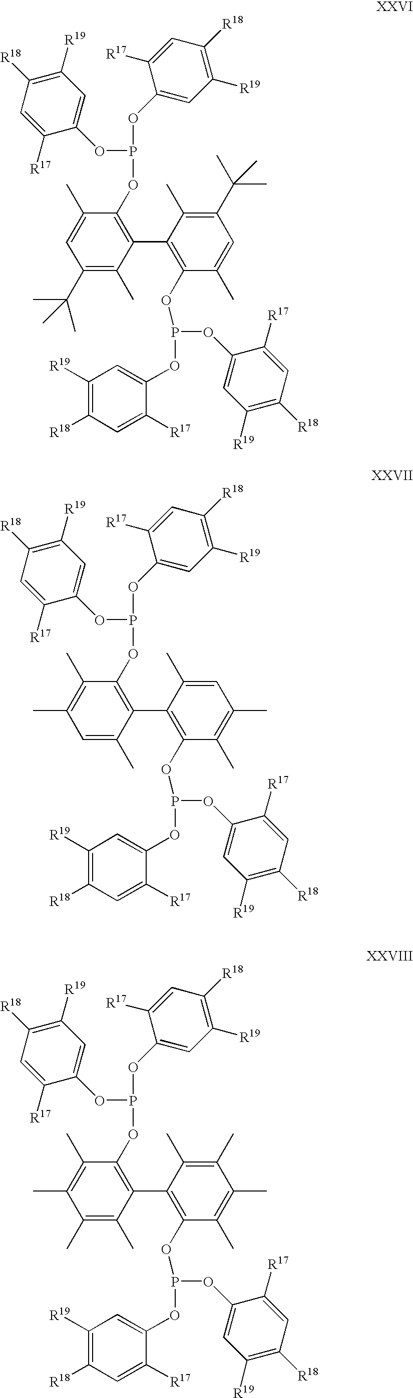

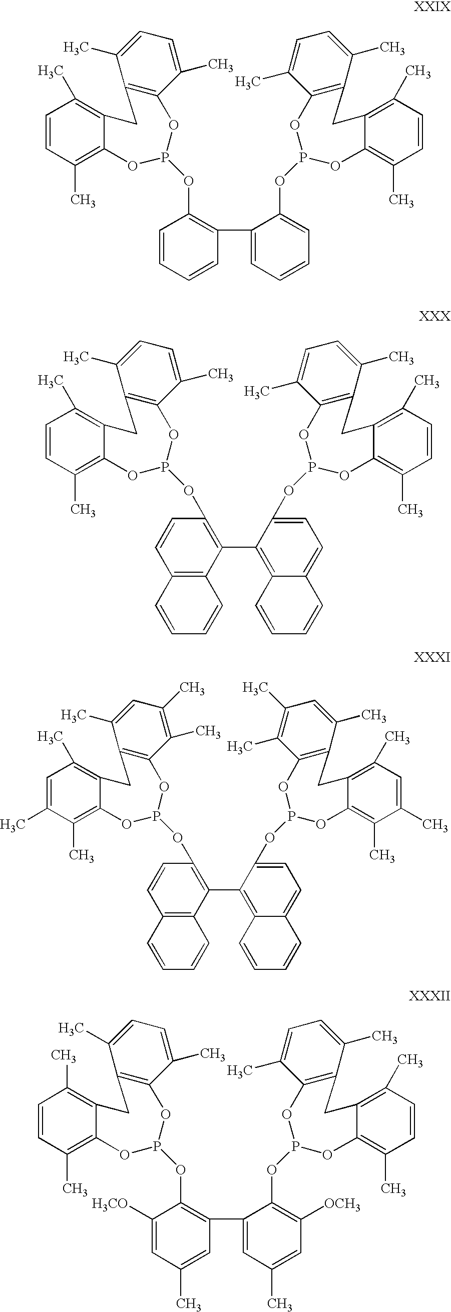

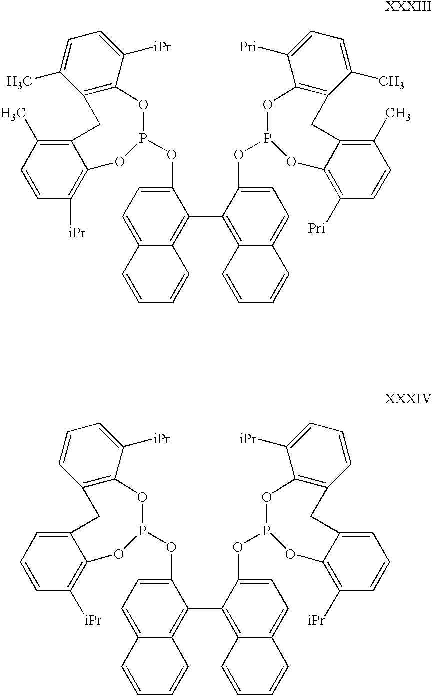

Suitable bidentate phosphites are of the type disclosed in U.S. Pat. Nos. 5,512,695; 5,512,696; 5,663,369; 5,688,986; 5,723,641; 5,959,135; 6,120,700; 6,171,996; 6,171,997; 6,399,534; the disclosures of which are incorporated herein by reference. Suitable bidentate phosphinites are of the type disclosed in U.S. Pat. Nos. 5,523,453 and 5,693,843, the disclosures of which are incorporated herein by reference.

The ratio of bidentate ligand to active nickel can vary from a bidentate ligand to nickel ratio of 0.5:1 to a bidentate ligand to nickel ratio of 100:1. Preferentially the bidentate ligand to nickel ratio ranges from 1:1 to 4:1.

The ligands in the present invention can also be multidentate with a number of phosphorous atoms in excess of 2 or of polymeric nature in which the ligand/catalyst composition is not homogeneously dissolved in the process mixture.

Optionally, the process of this invention is carried out in the presence of one or more Lewis acid promoters that affect both the activity and the selectivity of the catalyst system. The promoter may be an inorganic or organometallic compound in which the cation is selected from scandium, titanium, vanadium, chromium, manganese, iron, cobalt, copper, zinc, boron, aluminum, yttrium, zirconium, niobium, molybdenum, cadmium, rhenium and tin. Examples include but are not limited to ZnBr₂, ZnI₂, ZnCl₂, ZnSO₄, CuCl₂, CuCl, Cu(O₃SCF₃)₂, COCl₂, Col₂, FeI₂, FeCl₃, FeCl₂, FeCl₂(THF)₂, TiCl₄ (THF)₂, Cl₂Ti(OiPr)₂, MnCl₂, ScCl₃, AlCl₃, (C₈H₁₇)AlCl₂, (C₈H₁₇)₂AlCl, (iso-C₄H₉)₂AlCl, Ph₂AlCl, PhAlCl₂, ReCl₅, ZrCl₄, NbCl₅, VCl₃, CrCl₂, MOCl₅, YCl₃, CdCl₂, LaCl₃, Er(O₃SCF₃)₃, Yb(O₂CCF₃)₃, SmCl₃, B(C₆H₅)₃, R⁴⁰Sn(O₃SCF₃) where R⁴⁰ is an alkyl or aryl group. Preferred promoters include FeCl₂, ZnCl₂, COCl₂, Col₂, AlCl₃, B(C₆H₅)₃, and (C₆H₅)₃Sn(O₃SCF₃). The mole ratio of promoter to Group VIII transition metal present in the reaction can be within the range of about 1:16 to about 50:1, with 0.5:1 to about 2:1 being preferred.

The ligand compositions of the present invention may be used to form catalysts, which may be used for the hydrocyanation of the norbornene derivatives of the invention, with or without a Lewis acid promoter.

The process comprises contacting, in the presence of the catalyst, the norbornene derivative with a hydrogen cyanide-containing fluid under conditions sufficient to produce a nitrile. Any fluid containing about 1 to 100% HCN can be used. Pure hydrogen cyanide may be used.

The hydrocyanation process can be carried out, for example, by charging a suitable vessel, such as a reactor, with the norbornene derivative, catalyst composition, and optionally a solvent, to form a reaction mixture. Hydrogen cyanide can be initially combined with other components to form the mixture. However, it is preferred that HCN be added slowly to the mixture after other components have been combined. Hydrogen cyanide can be delivered as a liquid or as a vapor to the reaction. As an alternative, a cyanohydrin can be used as the source of HCN as known in the art.

Another suitable technique is to charge the vessel with the catalyst and the solvent (if any) to be used, and feed both the norbornene derivative and the HCN slowly to the reaction mixture.

The molar ratio of the norbornene derivative to catalyst can be varied from about 10:1 to about 100,000:1. The molar ratio of HCN catalyst can be from 5:1 to 10:000:1. The process can be run in continuous or batch mode.

Preferably, the reaction mixture is agitated, for example, by stirring or shaking. The present norbornane nitrile derivatives can be individually isolated from the reaction mixture, using known conventional methods, such as chromatography or fractional distillation or crystallization.

The hydrocyanation can be carried out with or without a solvent. The solvent, if used, can be liquid at the reaction temperature and pressure and inert towards the norbornene derivative and the catalyst. Examples of suitable solvents include hydrocarbons such as benzene, xylene, or combinations thereof; ethers such as tetrahydrofuran (THF); nitrites such as acetonitrile, adiponitrile, or combinations of two or more thereof. The norbornene derivative can itself serve as the solvent.

The exact temperature is dependent to a certain extent on the particular catalyst being used, and the desired reaction rate. Normally, temperatures of from −25° C. to 200° C. can be used, the range of about 0° C. to about 120° C. being preferred.

The process can be run at atmospheric pressures. Pressures of from about 50.6 to 1013 kPa are preferred. Higher pressures, up to 10,000 kPa or more, can be used, if desired.

The time required can be in the range of from a few seconds to many hours (such as 2 seconds to 72 hours), depending on the particular conditions and method of operation.

The norbornene derivative used as starting material in this invention contains a substituted norbornene (bicyclo[2.2.1]heptene) fragment which is hydrocyanated using the hydrocyanation process of this invention to the products of this invention, the norbornane nitrile derivatives. These substituted norbornene starting materials can be prepared using procedures known in the literature. Typical examples are described in Organic Chemistry, 3^{rd} Edition, Peter Vollhardt and Neil Schore, New York, Freeman and Company, 1998, pg 600, or in U.S. Pat. No. 5,861,528, U.S. Pat. No. 6,100,323, or U.S. Pat. No. 5,284,929.

In a first preferred embodiment, the present invention relates to compounds with the general structure of formula (XXXVI): 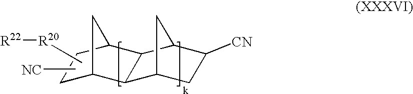
wherein k and R²⁰, R²¹ and R²² are as defined above.

The exact point of attachment and orientation of CN and R²⁰-R²² can vary and mixtures of compounds and isomers are commonly produced by this invention. Structure (XXXVI) is defined by structure (I) when A equals nothing, B equals CN and at least one of R²⁰-R²² is not H.

Preferred norbornane nitrile derivatives in this embodiment are for example structures (XXXVII-XLIV): 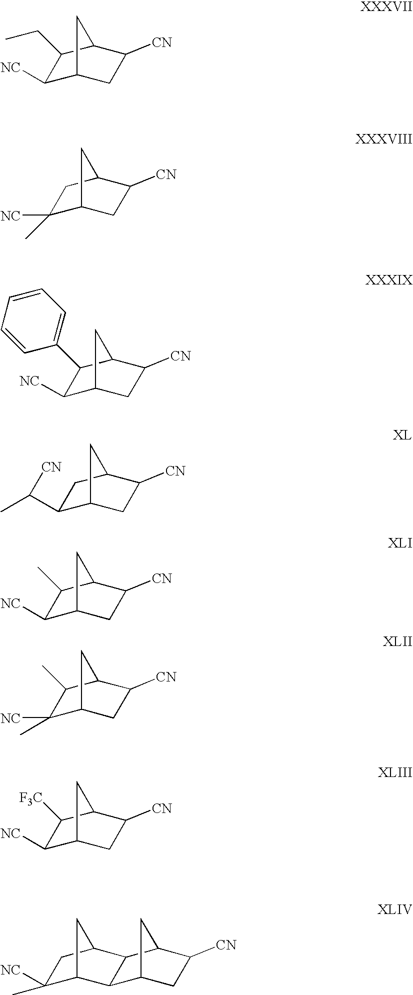
as a single isomer or as a mixture of isomers, or as a mixture of different compounds of structure (XXXVI).

For the production of the compounds of formula (XXXVI-XLIV), the norbornene derivative is reacted with hydrogen cyanide in the presence of a group VIII catalyst, preferably nickel, a ligand and optionally a promoter. In this embodiment, a product mixture is obtained which generally comprises norbornane derivatives having two nitrile groups.

In another preferred embodiment, the present invention relates to compounds with the general structure of formula (XLV): 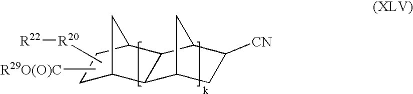
wherein k and R²⁰, R²¹, R²², and R²⁹ are as defined above.

The exact point of attachment and orientation of the —C(O)OR²⁹ group and the substituents R²⁰-R²² can vary and mixtures of compounds and isomers are commonly produced by this invention. Structure (XLV) is defined by structure (I) when A=nothing and B=C(O)OR²⁹. Preferred norbornane based nitrile derivatives in this embodiment are for example structures (XLVI-XLIX): 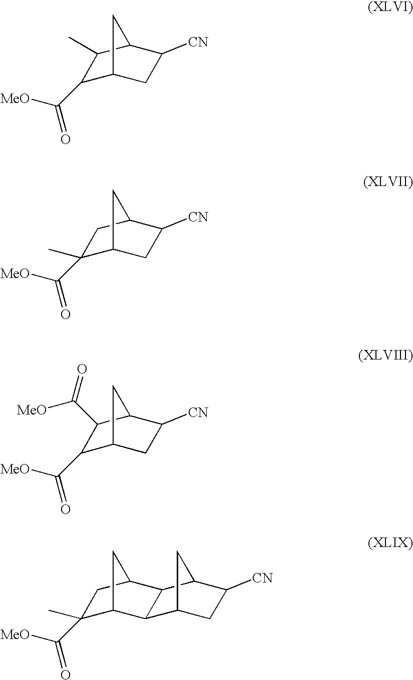
as a single isomer or as a mixture of isomers, or as a mixture of different compounds of structure (XLV).

For the production of the compounds of formula (XLVI-XLIX), the norbornene derivative is reacted with hydrogen cyanide in the presence of a group VIII catalyst, preferably nickel, a ligand and optionally a promoter. In this embodiment, a product mixture is obtained which generally comprises norbornane derivatives having one nitrile group and one or more ester groups.

In another preferred embodiment, the present invention relates to compounds with the general structure of formula (L-LIV):

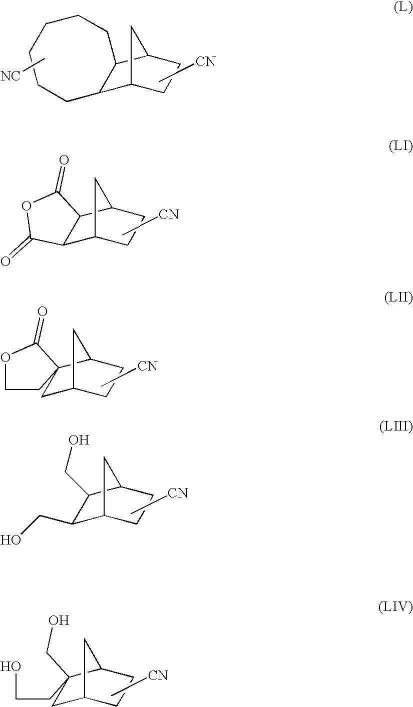

The exact point of attachment and orientation of the —CN group can vary and mixtures of compounds are commonly produced by this invention. Structure (L) is defined by structure (I) when A incorporates a ring that connects back to the norbornane skeleton and B equals —CN. Structures (LI) and (LII) are defined by structure (I) when A equals nothing, B equals C(O)OR²⁴ and R²⁴ connects back to the norbornane skeleton. Structure (LIII) is defined by structure (I) when A equals nothing, B equals CH₂OH, and R²⁰ equals CH₂OH. Structure (LIV) is defined by structure (I) when A equals nothing, B equals CH₂OH, and R²⁰ equals CH₂CH₂OH.

For the production of the compounds of formula (L-LIV), the norbornene derivative is reacted with hydrogen cyanide in the presence of a group VIII catalyst, preferably nickel, a ligand and optionally a promoter. In this embodiment, a product mixture is obtained which generally comprises norbornane derivatives having one or two nitrile groups and in case of (LI) an anhydride group and in case of (LII) a lactone group, in case of (LIII) and (LIV) a diol group.

It will be appreciated that the ester groups of (XLV)-(XLIX) and (LII) and the anhydride group of (LI) may be converted to alcohol groups by methods known in the art, e.g. reduction with hydride reagents (LiAlH₄) or catalytic ester hydrogenation.

In another preferred embodiment, the present invention relates to compounds with the general structure of formula (LV): 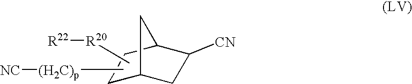
with one of the substituents R²⁰ to R²² is selected independently from the group hydrogen, methyl or other branched or linear alkyl groups and with p equal to an integer 1-12.

The exact point of attachment and orientation of the —(CH₂)ₚ—CN group and the substituents R²⁰-R²² can vary and mixtures of compounds and isomers are commonly produced by this invention. Structure (LV) is defined by structure (I) when A equals (CH₂)ₚ and B equals CN.

Preferred norbornane nitrile derivatives in this embodiment are for example structures (LVI-LVII):

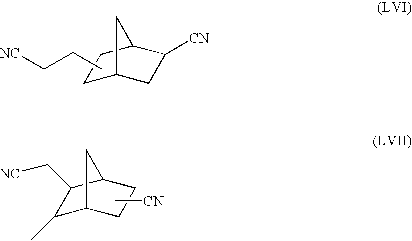

The exact point of attachment and orientation of the —CN group can vary and mixtures of compounds are commonly produced by this invention.

For the production of the compounds of formula (LVI-LVII), the norbornene derivative is reacted with hydrogen cyanide in the presence of a group VIII catalyst, preferably nickel, a ligand and optionally a promoter. In this embodiment, a product mixture is obtained which generally comprises norbornane derivatives having two nitrile groups.

In another preferred embodiment, the present invention relates to compounds with the structure of formulae (LVIII-LX):

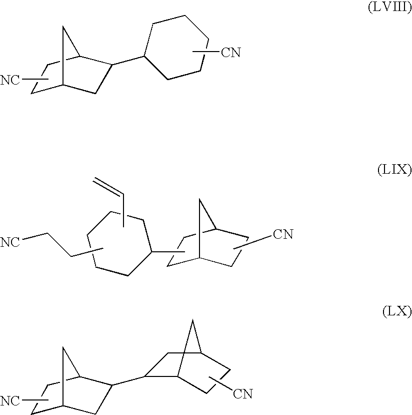

The exact point of attachment and orientation of the —CN group as well as the orientation of the two cycloaliphatic rings can vary and mixtures of compounds are commonly produced by this invention. Structures (LVIII), (LIX) and (LX) are defined by structure (I) when A equals a cycloaliphatic or substituted cycloaliphatic group that is not fused to the norbornane skeleton and B equals CN.

For the production of the compounds of formula (LVIII-LX), the norbornene derivative is reacted with hydrogen cyanide in the presence of a group VIII catalyst, preferably nickel, a ligand and optionally a promoter. In this embodiment, a product mixture is obtained which generally comprises norbornane derivatives having one or two nitrile groups.

Having generally described this invention, a further understanding can be obtained by reference to certain specific examples, which are provided herein for purpose of illustration only and are not intended to be limiting.

### EXAMPLES

The ligands LXI, LXII, LXIII, LXIV were used for the hydrocyanation reactions described in these examples.

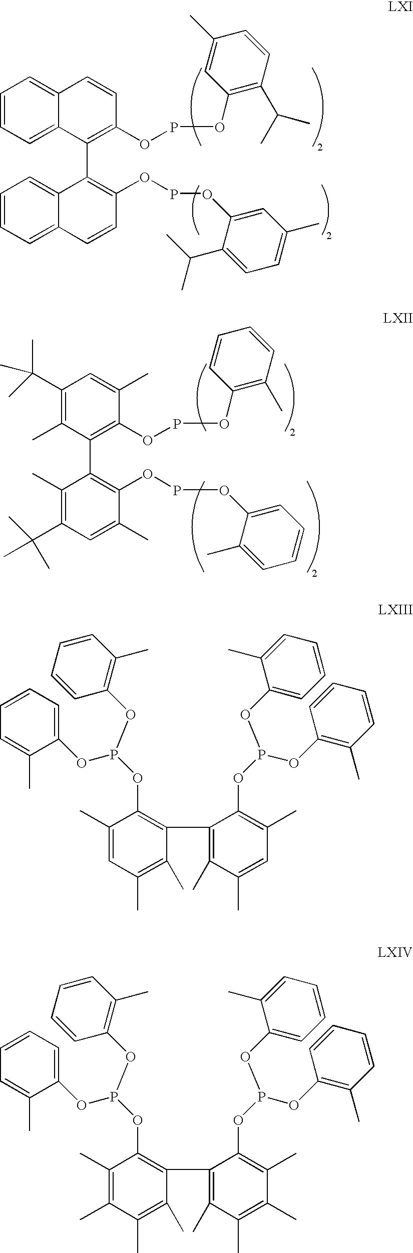

### Example 1

In a 500 ml flask 3-ethyl-bicyclo[2.2.1]hept-5-ene-2-carbonitrile (114 g, 0.78 mol) was mixed with a toluene (5 g) solution of Ni(COD)₂ (0.7 g, 2.6 mmol) and ligand (LXI) (2.9 g, 3.1 mmol). To this was added a solution of ZnCl₂ (0.35 g, 2.6 mmol) in acetonitrile (5 g). A solution of hydrogen cyanide (25 g, 0.9 mol) in acetonitrile (38 g) was prepared and added to the above mixture using a syringe pump. After 21 hours reaction time at 50° C. the product compound (XXXVII) was formed in 98.7% yield. Product composition was analyzed using standard GC methodology.

### Example 2

In a 1000 ml flask 2-methyl-5-norbornene-2-carbonitrile (542, 4.1 mol) was mixed with a toluene (45 g) solution of Ni(COD)₂ (2.24 g, 8.1 mmol) and ligand (LXI) (9.2 g, 9.8 mmol). To this was added a solution of ZnCl₂ (1.1 g, 8.1 mmol) in acetonitrile (11 g). A solution of hydrogen cyanide (110 g, 4.1 mol) in acetonitrile (164 g) was prepared and added to the above mixture using a syringe pump. After 15 hours reaction time at 70° C. the product compound (XXXVIII) was formed essentially quantitatively. Product composition was analyzed using standard GC methodology.

### Example 3

In a 500 ml flask 3-(trifluoromethyl)-5-norbornene-2-carbonitrile (14.5 g, 0.1 mol) was mixed with a toluene (5 g) solution of Ni(COD)₂ (0.26 g, 0.95 mmol) and ligand (LXIII) (0.98 g, 1.3 mmol). To this was added a solution of ZnCl₂ (0.14 g, 1.05 mmol) in acetonitrile (5 g). A solution of hydrogen cyanide (2.6 g, 0.1 mol) in acetonitrile (3.7 g) was prepared and added to the above mixture using a syringe pump. After 9.5 hours reaction time at 50° C. the product compound (XLIII) was formed essentially quantitatively. Product composition was analyzed using standard GC methodology.

### Example 4

In a 1000 ml flask 2-methyl-1,2,3,4,4a,5,8,8a-octahydro-1,4:5,8-dimethanonaphthalene-2-carbonitrile (522 g, 2.6 mol, prepared as the dominant component in a Diels Alder reaction of excess dicyclopentadiene and methacrylonitrile) was mixed with a toluene (45 g) solution of Ni(COD)₂ (2.16 g, 7.8 mmol) and ligand (LXII) (9.24 g, 11 mmol). To this was added a solution of ZnCl₂ (1.1 g, 7.8 mmol) in acetonitrile (20 g). A solution of hydrogen cyanide (69 g, 2.6 mol) in acetonitrile (104 g) was prepared and added to the above mixture using a syringe pump over time. After 8 hours addition time at 85° C. the product compound (XLIV) was formed next to two byproducts in 70% yield. The two byproducts are compound (XXXVIII) and a product derived from a cyclopentadiene oligomer generated in the Diels Alder reaction. The product composition was analyzed using standard GC methodology. The desired compound was isolated in a fractional distillation with a purity of 97%.

### Example 5

In a 1000 ml flask 5-methyl-5-(methoxycarbonyl)bicyclo[2.2.1]hept-2-ene (214 g, 1.3 mol) was mixed with Ni(COD)₂ (0.71 g, 2.6 mmol) and ligand (LXI) (2.91 g, 3 mmol). To this was added ZnCl₂ (0.35 g, 2.6 mmol). A solution of hydrogen cyanide (33 g, 1.2 mol) in acetonitrile (49.6 g) was prepared and added to the above mixture using a syringe pump. After 290 minutes addition time at 50° C. the product compound (XLVII) was formed with a yield of 96.7%. Product composition was analyzed using standard GC methodology.

### Example 6

In a 500 ml flask 3-methyl-bicyclo[2.2.1]hept-5-ene-2-carboxylic acid-methyl ester.

(125, 0.75 mol) was mixed with a toluene (30 g) solution of Ni(COD)₂ (0.7 g, 2.5 mmol) and ligand (LXI) (2.95 g, 3.5 mmol). To this was added a solution of ZnCl₂ (0.34 g, 2.5 mmol) in acetonitrile (10 g). A solution of hydrogen cyanide (20 g, 0.73 mol) in acetonitrile (30 g) was prepared and added to the above mixture using a syringe pump. After 3 hours reaction time at 50° C. the product compound (XLVI) was formed with a 94% yield. Product composition was analyzed using standard GC methodology.

### Example 7

In a 500 ml flask the dimethyl ester of bicyclo[2.2.1]hept-5-ene-2,3-dicarboxylic acid (564 g, 0.75 mol) was mixed with a toluene (30 g) solution of Ni(COD)₂ (1.5 g, 5.4 mmol) and ligand (LXI) (5.56 g, 5.4 mmol). To this was added a solution of ZnCl₂ (0.73 g, 5.4 mmol) in acetonitrile (20 g). A solution of hydrogen cyanide (70 g, 2.6 mol) in acetonitrile (105 g) was prepared and added to the above mixture using a syringe pump. After 5 hours reaction time at 50° C. the product compound (XLVIII) was formed with a 97.4% yield. Product composition was analyzed using standard GC methodology.

### Example 8

In a 100 ml flask 2-(hydroxymethyl)-bicyclo[2.2.1]hept-5-ene-2-ethanol (38.4 g, 0.23 mol) was mixed with a toluene (20 g) solution of Ni(COD)₂ (0.16 g, 0.57 mmol) and ligand (LXII) (0.67 g, 0.8 mmol). To this was added a solution of ZnCl₂ (0.08 g, 0.57 mmol) in acetonitrile (10 g). A solution of hydrogen cyanide (6 g, 0.22 mol) in acetonitrile (9 g) was prepared and added to the above mixture using a syringe pump. After 7 hours reaction time at 50° C. the product compound (LIV) was formed with a 99.7% yield. Product composition was analyzed using standard GC methodology.

### Example 9

In a 100 ml flask 1,4,4a,5,6,9,10,10a-octahydro-1,4-methanobenzocyclooctene, (72 g, 0.41 mol) was mixed with a toluene (5 g) solution of Ni(COD)₂ (0.57 g, 2.1 mmol) and ligand (LXI) (2.34 g, 2.5 mmol). To this was added a solution of ZnCl₂ (0.28 g, 2.1 mmol) in acetonitrile (10 g). A solution of hydrogen cyanide (13.4 g, 0.5 mol) in toluene (53.7 g) was prepared and added to the above mixture using a syringe pump. After 21 hours reaction time at 50° C. the starting material has been converted with 96.4%. The dinitrile product (L) was formed with 53.4% yield, the remainder is the mono-nitrile addition product. Product composition was analyzed using standard GC methodology.

### Example 10

In a 1000 ml flask carbic anhydride, (50 g, 0.30 mol) was dissolved into tetrahydrofuran (100 g). To this was added a solution of Ni(COD)₂ (0.17 g, 0.61 mmol) and ligand (LXII) (0.7 g, 0.82 mmol). To this was added a solution of ZnCl₂ (0.09 g, 0.67 mmol) in acetonitrile (5 g). A solution of hydrogen cyanide (7.4 g, 0.27 mol) in acetonitrile (11.1 g) was prepared and added to the above mixture using a syringe pump while the internal temperature did not exceed 50° C. After 3 hours reaction time product (LI) was formed with 48.5% yield. Product composition was analyzed using standard GC methodology.

### Example 11

In a 500 ml flask 5-(3,4-diethenylcyclohexyl)-bicyclo[2.2.1]hept-2-ene, (50 g, 0.22 mol) was mixed with a toluene (10 g) solution of Ni(COD)₂ (0.30 g, 1.1 mmol) and ligand (LXI) (1.4 g, 1.48 mmol). To this was added a solution of ZnCl₂ (0.16 g, 1.20 mmol) in acetonitrile (5 g). A solution of hydrogen cyanide (5.33 g, 0.22 mol) in acetonitrile (8 g) was prepared and added to the above mixture using a syringe pump while the internal temperature did not exceed 50° C. After 12 hours reaction time product (LIX) was formed in 8% yield, while the product 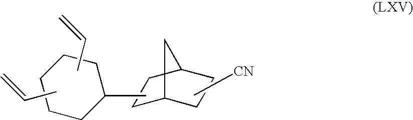
was formed in 65% yield. Product composition was analyzed using standard GC methodology.

### Example 12

In a 1000 ml flask 5-(3-cyclohexen-1-yl)-bicyclo[2.2.1]hept-2-ene, (140 g, 0.80 mol) was mixed with a toluene (10 g) solution of Ni(COD)₂ (1.1 g, 4.0 mmol) and ligand (LXI) (4.55 g, 4.82 mmol). To this was added a solution of ZnCl₂ (0.55 g, 4.0 mmol) in acetonitrile (5 g). A solution of hydrogen cyanide (26.1 g, 0.97 mol) in acetonitrile (39.2 g) was prepared and added to the above mixture using a syringe pump at 50° C. After 11 hours reaction time the starting material was converted to 99.7% with the formation of a mononitrile adduct with 75.4% yield and the formation of product (LVIII) with 24.3% yield. Product composition was analyzed using standard GC methodology.

### Example 13

In a 1000 ml flask 5-ethenyl-bicyclo[2.2.1]hept-2-ene, (105 g, 0.87 mol) was mixed with a toluene (10 g) solution of Ni(COD)₂ (1.12 g, 4.38 mmol) and ligand (LXI) (4.94 g, 5.24 mmol). To this was added a solution of ZnCl₂ (0.65 g, 4.8 mmol) in acetonitrile (5 g). A solution of hydrogen cyanide (27.15 g, 1.0 mol) in acetonitrile (40.7 g) was prepared and added to the above mixture using a syringe pump at 50° C. After 19 hours reaction time the starting material was converted to 98.6% with the formation of a mononitrile adduct with 34.6% yield and the formation of product (LVI) with 64.0% yield. Product composition was analyzed using standard GC methodology.

### Example 14

In a 500 ml flask 2,3-dimethanol-bicyclo[2.2.1]hept-5-ene, (42.8 g, 0.28 mol) was mixed with a toluene (10 g) solution of Ni(COD)₂ (0.38 g, 1.39 mmol) and ligand (LXI) (1.77 g, 1.87 mmol). To this was added a solution of ZnCl₂ (0.21 g, 1.53 mmol) in acetonitrile (5 g). A solution of hydrogen cyanide (7.5 g, 0.28 mol) in tetrahydrofuran (11.3 g) was prepared and added to the above mixture using a syringe pump at 50° C. After 15 hours reaction time the formation of product (LIII) was observed with essentially quantitative yield. Product composition was analyzed using standard GC methodology.

### Example 15

In a 500 ml flask methyl 4-methyltetracyclo[6.2.1.13,6.02,7]dodec-9-ene-4-carboxylate, (50 g, 0.22 mol) was mixed with a toluene (5 g) solution of Ni(COD)₂ (0.12 g, 0.43 mmol) and ligand (LXI) (0.55 g, 0.58 mmol). To this was added a solution of ZnCl₂ (0.065 g, 0.47 mmol) in acetonitrile (5 g). Hydrogen cyanide (5.2 g, 0.19 mol) was added to the above mixture using a syringe pump at 50° C. The reaction was started at room temperature but showed an exotherm and the reaction temperature reached 80° C. After 2 hours reaction time the formation of product (XLIX) was observed with a 85% yield. The product composition was analyzed using standard GC methodology.

### Example 16

In a 500 ml flask 5,5′-bibicyclo[2.2.1]hept-2-ene, (20 g, 0.17 mol) was mixed with a toluene (5 g) solution of Ni(COD)₂ (0.09 g, 0.33 mmol) and ligand (LXI) (0.38 g, 0.4 mmol). To this was added a solution of ZnCl₂ (0.05 g, 0.33 mmol) in acetonitrile (5 g). Hydrogen cyanide (9.4 g, 0.35 mol) was added to the above mixture using pipette addition. The reaction showed an exotherm and the reaction temperature reached 80° C. After one hour reaction time the formation of product (LX) was observed with a 15% yield next to 60% mononitrile products. The product mixture also contains side products generated in the Diels Alder reaction of vinyl-norbornene with dicyclopentadiene which can be hydrocyanated to nitrile products. The product composition was analyzed using standard GC methodology.

### Example 17

In a 500 ml flask 4′,5′-dihydro-spiro[bicyclo[2.2.1]hept-5-ene-2,3′(2′H)-furan]-2′-one, (25 g, 0.152 mol) was mixed with a toluene (20 g) solution of Ni(COD)₂ (0.21 g, 0.76 mmol) and ligand (LXIV) (0.81 g, 1.03 mmol). To this was added a solution of ZnCl₂ (0.11 g, 0.76 mmol) in acetonitrile (5 g). A solution of hydrogen cyanide (4.12 g, 0.152 mol) in acetonitrile (6.2 g) was prepared and added to the above mixture using a syringe pump. The internal temperature did not exceed 50° C. After 16 hours reaction time product (LII) was formed with 81% yield. Product composition was analyzed using standard GC methodology.

### Example 18

In a 500 ml, 3-phenyl-bicyclo[2.2.1]hept-5-ene-2-carbonitrile, (15 g, 0.076 mol) was mixed with a toluene (20 g) solution of Ni(COD)₂ (0.14 g, 0.56 mmol) and ligand (LXIII) (0.52 g, 0.68 mmol). To this was added a solution of ZnCl₂ (0.08 g, 0.56 mmol) in acetonitrile (5 g). A solution of hydrogen cyanide (2.08 g, 0.077 mol) in acetonitrile (3.11 g) was prepared and added to the above mixture using a syringe pump while the internal temperature did not exceed 50° C. After 16 hours reaction time product (XXXIX) was formed with 70% yield. Product composition was analyzed using standard GC methodology.

### Example 19

In a 20 ml glass vessel, bicyclo[2.2.1]hept-5-ene-2-(α-methyl)-acetonitrile, (1.4 g, 9.5 mmol) was mixed with a toluene (0.5 g) solution of Ni(COD)₂ (0.01 g, 0.05 mmol) and ligand (LXI) (0.05 g, 0.068 mmol). To this was added a solution of ZnCl₂ (0.01 g, 0.05 mmol) in acetonitrile (0.5 g). A solution of hydrogen cyanide (0.26 g, 9.51 mmol) in acetonitrile (0.39 g) was prepared and added to the above mixture using an addition rate of one drop during two minutes. The internal temperature did not exceed 33° C. during this process. After 16 hours reaction time product (XL) was formed with 70% yield. Product composition was analyzed using standard GC methodology.

### Example 20

In a 500 ml flask 3-methyl-bicyclo[2.2.1]hept-5-ene-2-carbonitrile (33 g, 0.25 mol) was mixed with a toluene (10 g) solution of Ni(COD)₂ (0.14 g, 0.5 mmol) and ligand (LXI) (0.51 g, 0.55 mmol). To this was added a solution of ZnCl₂ (0.07 g, 0.55 mmol) in acetonitrile (5 g). A solution of hydrogen cyanide (6.7 g, 0.25 mol) in acetonitrile (10 g) was prepared and added to the above mixture using a syringe pump. After 2.5 hours reaction time at a self-sustained internal temperature of 45° C. the product compound (XLI) was formed in 85% yield. Product composition was analyzed using standard GC methodology.

### Examples 21-25

Amine derivatives of the norbornane nitrile derivatives of this invention were reacted with a typical epoxy resin to prepare films. Examples 21-25 were carried out using the di-amine derivatives prepared by hydrogenation of the norbornane nitrile derivatives of this invention.

Bis(4-glycidyloxyphenyl)methane (Aldrich) was placed in a reaction vial. To this was added the di-amine derived from the dinitriles of this invention in a mol ratio of 2:1 at room temperature. This mixture was mixed using a Vortex mixer for 2 minutes. The homogenous clear mixture was drawn out onto a glass plate and placed into the dry time recorder. The dry time recorder was set to a 24 hour cycle and the measurement was carried out at room temperature.

| | Nitrile | BK Drying Recorder | | | | | | | |
|---|---|---|---|---|---|---|---|---|---|
| Example Number | Derivative | Stage 0 | Stage 1 | | Stage 2 | | Stage 3 | | Stage 4 |
| 21 | XXXVII | 1 hr | 1-1.5 | hr | 1.5-2.5 | hr | 2.5-5 | hr | >8 hr |
| 22 | XXXVIII | 1.5 hr | 1.5-2.25 | hr | 2.25-3.25 | hr | 3.25-8 | hr | >14 hr |
| 23 | XLIV | 0.5 hr | 0.5-1.5 | hr | 1.5-2.25 | hr | 2.25-10 | hr | >14 hr |
| 24 | LVI | 1 hr | 1-2 | hr | 2-3 | hr | 3-10 | hr | >12 hr |
| 25 | LVIII | 1.5 hr | 1.5-3.5 | hr | 3.5-4 | hr | 4-6 | hr | >8 hr |

Stage 0: leveling,
Stage 1: basic trace,
Stage 2: film building,
Stage 3: Surface trace;
Stage 4: dry

Various modifications, alterations, additions or substitutions to the processes and compositions of this invention will be apparent to those skilled in the art without departing from the spirit and scope of this invention. This invention is not limited to the illustrative embodiments set forth herein, but rather is defined by the following claims.

1. A nitrile composition of formula (I) or mixtures or isomers thereof: 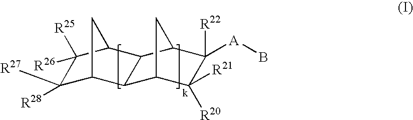
wherein
k equals 0, 1 or 2 and the bridging CH₂ group may be on the same or opposite side with respect to the first bridging CH₂ group,
wherein
R²⁰, R²¹, R²² can be the same or different and are each independently H, a C₁ to C₂₀ alkyl group, a C₁ to C₂₀ alkyl group substituted with a hydroxyl, a C₁ to C₁₈ perfluoroalkyl group, a phenyl group, an C₆ to C₂₀ aryl group substituted with a C₁-C₁₂ alkyl group, an C₆ to C₂₀ aryl group substituted with a hydroxyl group, a C(O)OR²⁹ group (with R²⁹ selected to be a C₁ to C₂₀ linear or branched or cyclic alkyl or C₆ to C₂₀ aryl group), or an alkylene chain (—(CH₂)_{q}—; q equals an integer 0-16) or nothing (in which case A or B may connect back to the norbornane skeleton) and
wherein
A equals nothing or any alkylene chain (—(CH₂)ₚ—; p equals an integer 1-16), any substituted C₁ to C₂₀ alkylene group (provided the substituent does not comprise a cyano group or an amino group and does not interfere with the process of this invention), a C₁ to C₂₀ cycloaliphatic group, or a C₁ to C₁₈ perfluoroalkylene group, and wherein A may form a ring of greater than 5 carbons that connects to the norbornane skeleton through R²⁰, R²¹ or R²²
with the proviso that R²⁰, R²¹ or R²² cannot all be H if A equals nothing and
wherein
B equals —CN, —(CH₂)ₛOH or —C(O)OR²⁴
with s equal to an integer 0-12 and with R²⁴ selected to be H, a C₁ to C₂₀ linear or branched or cyclic alkyl or alkylene group, a C₆ to C₂₀ aryl group or a C₁ to C₁₈ perfluorinated alkyl group and wherein R²⁴ may connect to the norbornane skeleton through R²⁰, R²¹ or R²²
or wherein
R²⁴ may equal a —C(O)— group which connects to the norbornane skeleton through R²⁰, R²¹ or R²² forming a cyclic anhydride and
wherein
R²⁵, R²⁶, R²⁷, R²⁸ can be the same or different and are each independently H or —CN, with the proviso that only one of R²⁵, R²⁶, R²⁷, R²⁸ is —CN.

2. The nitrile composition according to claim 1 of structure (I) 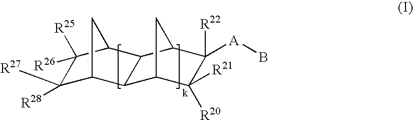
wherein
k equals 0 or 1 and A equals nothing and B is selected independently from the groups
—C(O)OR²⁹, or —CN,
while at least one of R²⁰-R²² is selected independently from methyl, ethyl, or a C₁ to C₂₀ linear or branched alkyl group or a C₁ to C₁₈ perfluoroalkyl group or a phenyl group or a C₆-C₂₀ aryl group substituted with a C₁ to C₂₀ linear or branched alkyl group or a C₆ to C₂₀ aryl group substituted with a hydroxyl, or a —C(O)OR²⁹ group,
with R²⁹ selected to be a C₁ to C₂₀ linear or branched or cyclic alkyl group or a C₆ to C₂₀ aryl group; and
one of the substituents R²⁵ to R²⁸ independently is —CN, while the other three substituents within the group R²⁵ to R²⁸ are hydrogen.

3. The nitrile composition according to claim 1 of structure (I) 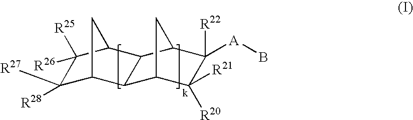
wherein k equals 0 or 1 and A equals nothing and B plus one of the substituents R²⁰ to R²² are selected to form an intramolecular cyclic anhydride or a lactone:
—(CH₂)ᵣC(O)OC(O)(CH₂)_{q}—,
—(CH₂)ᵣ—C(O)O—(CH₂)_{q}—,
with r and q equal to 0, 1, 2, 3, 4, 5 or 6, and
one of the substituents R²⁵ to R²⁸ independently is —CN, while the other three substituents within the group R²⁵ to R²⁸ are hydrogen.

4. The nitrile composition according to claim 1 of structure (I) 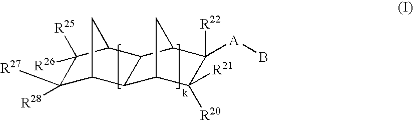
wherein k equals 0 or 1 and A equals nothing and
B equals —CH₂OH, R²⁰ equals —CH₂OH or —CH₂CH₂OH and
one of the substituents R²⁵ to R²⁸ independently is —CN, while the other three substituents within the group R²⁵ to R²⁸ are hydrogen.

5. The nitrile composition according to claim 1 of structure (I) 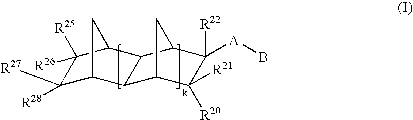
wherein k equals 0 or 1 and A and one of the substituents R²⁰ to R²² are selected to form a substituted cyclic aliphatic group with B attached thereto,
—(CH₂)ᵣCH(B)(CH₂)_{q}—,
with r and q each equal to an integer 0-15 and wherein 2<(r+q)<15
with B equal to a cyano group (—CN); and one of the substituents R²⁵ to R²⁸ independently is —CN, while the other three substituents within the group R²⁵ to R²⁸ are hydrogen.

6. The nitrile composition according to claim 1 of structure (I) 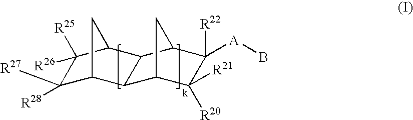
wherein
k equals 0 or 1, A equals —(CH₂)ₚ— and B equals —CN,
with p equal to an integer 1-12, while the substituents R²⁰ to R²² are hydrogen, methyl or a C₂ to C₂₀ branched or linear alkyl groups; and
one of the substituents R²⁵ to R²⁸ independently is —CN, while the other three substituents within the group R²⁵ to R²⁸ are hydrogen.

7. The nitrile composition according to claim 1 of structure (I) 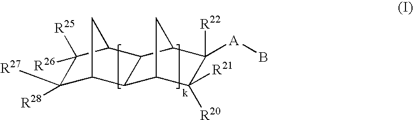
wherein
A is selected from
a substituted cyclohexyl group 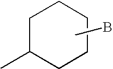
or a substituted vinyl cyclohexyl group 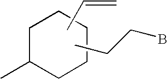
or a substituted norbornyl group 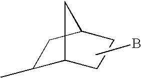
while R²⁰ to R²² are hydrogen, B=—CN; and
wherein one of the substituents R²⁵ to R²⁸ independently is —CN while the other three substituents within the group R²⁵ to R²⁸ are hydrogen.

8. A hydrocyanation process for the preparation of substituted norbornane nitrile compounds of claim 1 comprising contacting a corresponding substituted norbornene compound with a hydrogen cyanide-containing fluid, in the presence of a catalyst, to produce a nitrile composition of formula (I)

9. The process of claim 8 wherein the catalyst comprises an organic phosphorus ligand and a Group VIII metal or compound.

10. The process of claim 9 wherein the Group VIII metal or compound is selected from the group consisting of nickel, cobalt, and palladium.

11. The process of claim 10 wherein the organic phosphorous ligand is independently selected from the group consisting of monodentate and bidentate phosphite ligands of structural formulae II, III, IV, and V: 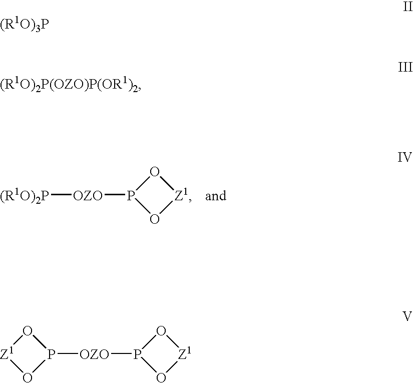
wherein R¹ is phenyl, unsubstituted or substituted with one or more C₁ to C₁₂ alkyl or C₁ to C₁₂ alkoxy groups; or naphthyl, unsubstituted or substituted with one or more C₁ to C₁₂ alkyl or C₁ to C₁₂ alkoxy groups; and Z and Z¹ are independently selected from the group consisting of structural formulae VI, VII, VIII, IX, and X: 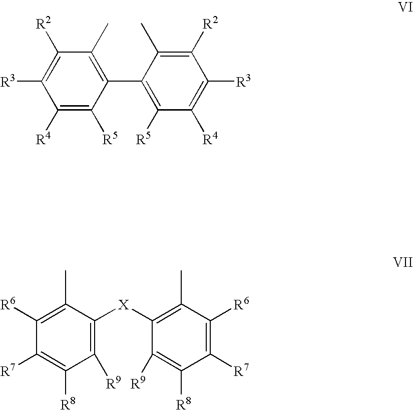
wherein
R², R³, R⁴, R⁵, R⁶, R⁷, R⁸, and R⁹ are independently selected from H, C₁ to C₁₂ alkyl, and C₁ to C₁₂ alkoxy;
X is O, S, or CH(R¹⁰); R¹⁰ is H or C₁ to C₁₂ alkyl; 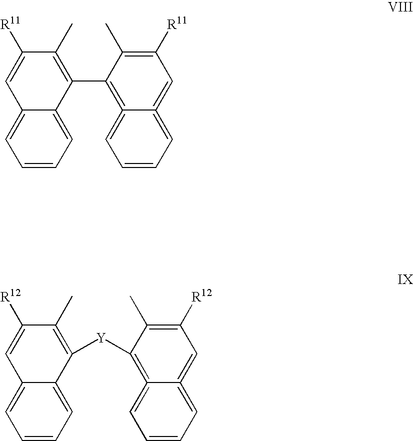
wherein
R¹¹ and R¹² are independently selected from H, C₁ to C₁₂ alkyl, and C₁ to C₁₂ alkoxy; and CO₂R¹³
R¹³ is C₁ to C₁₂ alkyl or C₆ to C₁₀ aryl, unsubstituted or substituted. with C₁ to C₄ alkyl
Y is O, S, CH(R¹⁴);
R¹⁴ is H or C₁ to C₁₂ alkyl 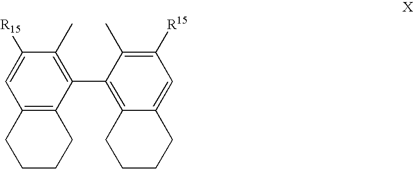
wherein
R¹⁵ is selected from H, C₁ to C₁₂ alkyl, and C₁ to C₁₂ alkoxy; and CO₂R¹⁶,
R¹⁶ is C₁ to C₁₂ alkyl or C₆ to C₁₀ aryl, unsubstituted or substituted with C₁ to C₄ alkyl.

12. The process of claim 11 wherein the ligand is a bidentate phosphite ligand independently selected from the group consisting of structural formulae XI to XXXIV: 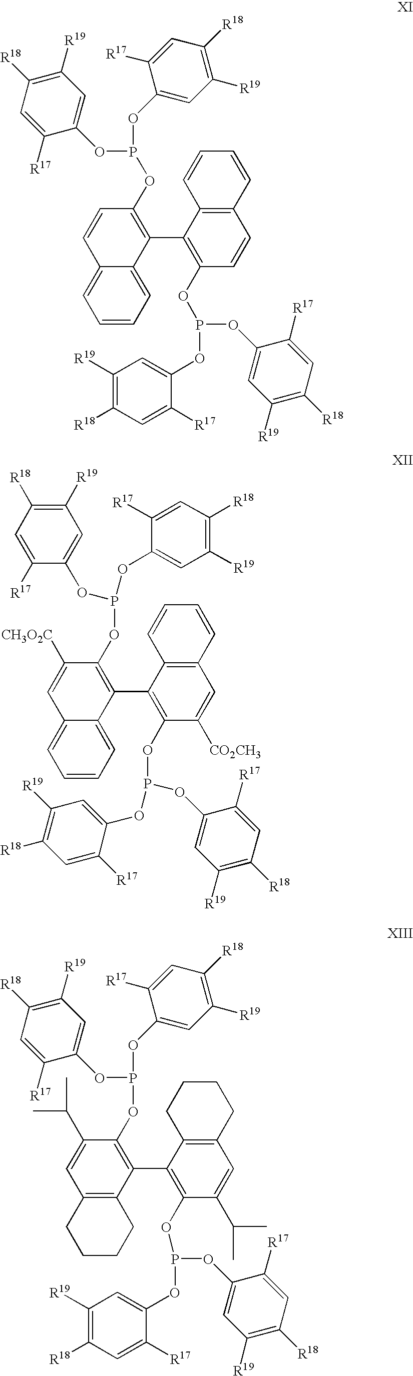 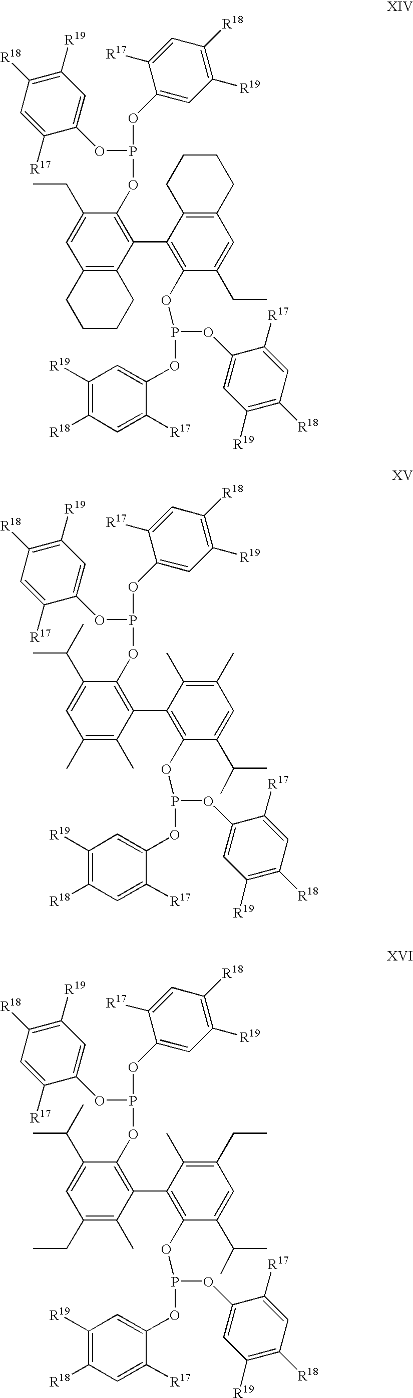 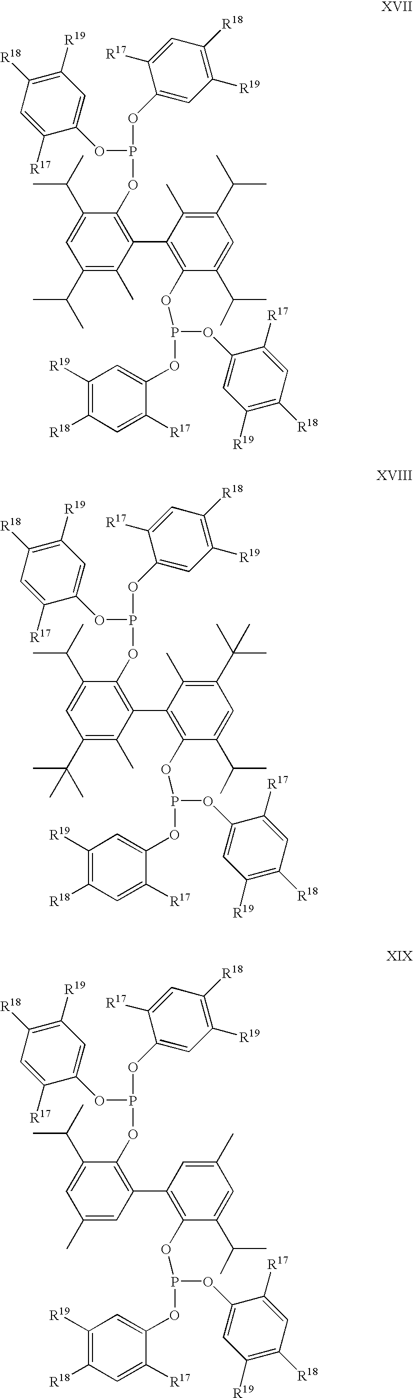 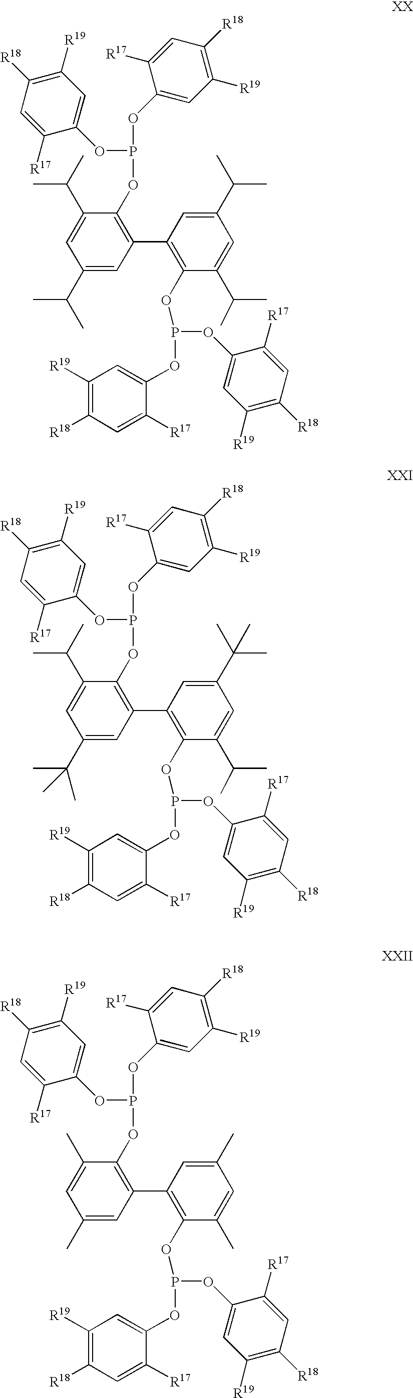 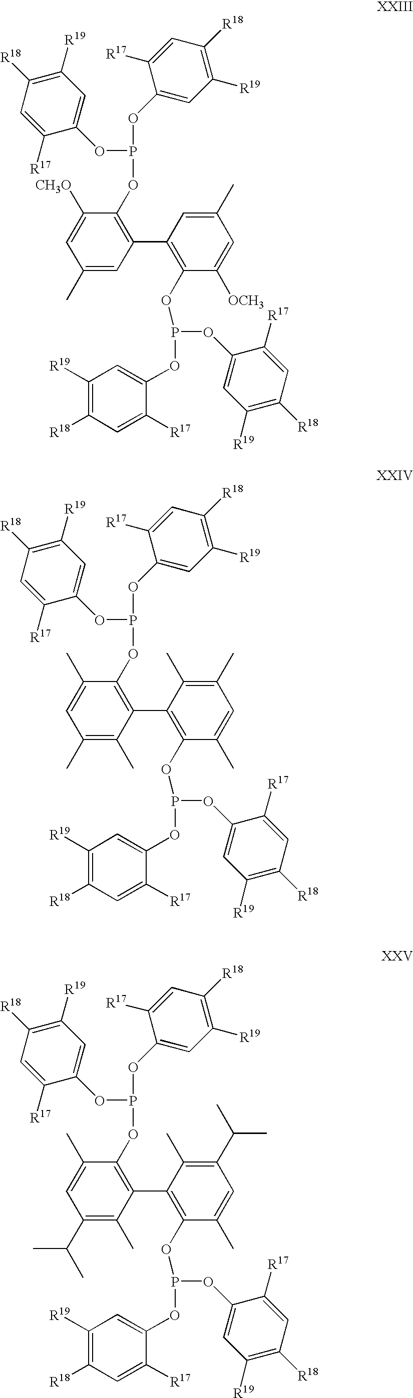 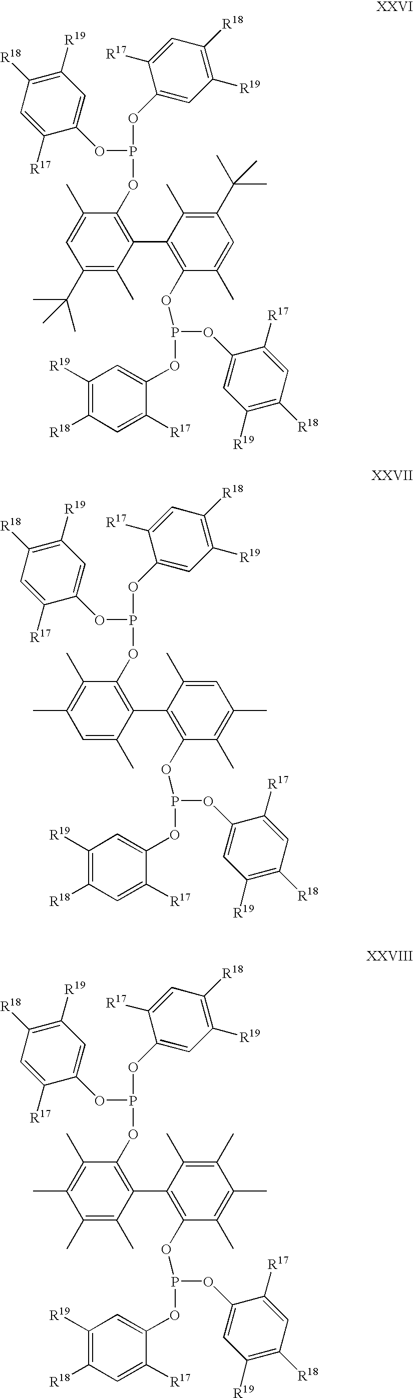 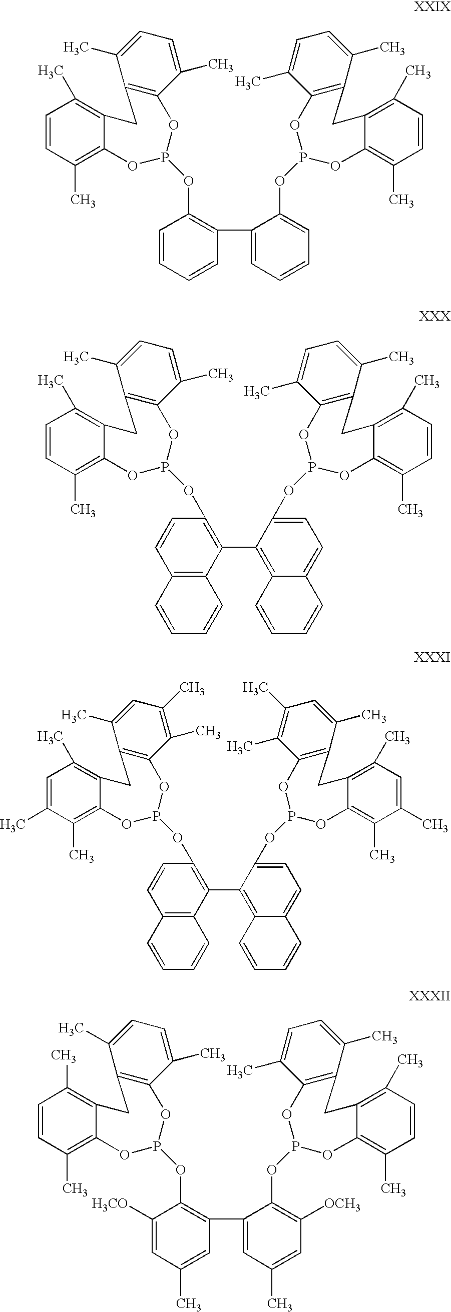 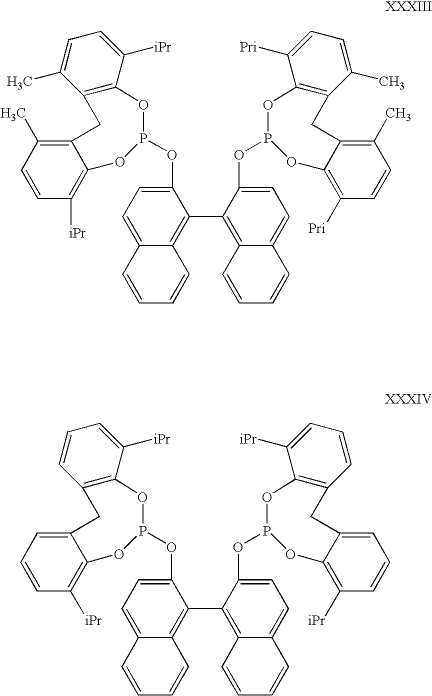
wherein for each formula, R¹⁷ is selected from the group consisting of H, methyl, ethyl or isopropyl, and R¹⁸ and R¹⁹ are independently selected from H or methyl.

13. The process of claim 8 conducted in the presence of a solvent.

14. The process of claim 8 conducted in the presence of a promoter.
